SEQUENCE LISTING (17 sequences)

SEQ ID NO: 1 - protein, 3 aa, Artificial Sequence
  Description of Artificial Sequence Synthetic peptide
RGD

SEQ ID NO: 2 - protein, 15 aa, Artificial Sequence
  Description of Artificial Sequence Synthetic construct
RSKAKWQTGTNPLYR

SEQ ID NO: 3 - protein, 15 aa, Artificial Sequence
  Description of Artificial Sequence Synthetic construct
RARAKWDTANNPLYK

SEQ ID NO: 4 - protein, 15 aa, Artificial Sequence
  Description of Artificial Sequence Synthetic construct
RSRARYEMASNPLYR

SEQ ID NO: 5 - protein, 16 aa, Artificial Sequence
  Description of Artificial Sequence Synthetic construct
KEKLKSQWNNDNPLFK

SEQ ID NO: 6 - protein, 10 aa, Artificial Sequence
  Description of Artificial Sequence Synthetic construct
RSKAKNPLYR

SEQ ID NO: 7 - protein, 10 aa, Artificial Sequence
  Description of Artificial Sequence Synthetic construct
RARAKNPLYK

SEQ ID NO: 8 - protein, 10 aa, Artificial Sequence
  Description of Artificial Sequence Synthetic construct
RSRARNPLYR

SEQ ID NO: 9 - protein, 10 aa, Artificial Sequence
  Description of Artificial Sequence Synthetic construct
KEKLKNPLFK

SEQ ID NO: 10 - protein, 5 aa, Artificial Sequence
  Description of Artificial Sequence Synthetic peptide
WQTGT

SEQ ID NO: 11 - protein, 6 aa, Artificial Sequence
  Description of Artificial Sequence Synthetic peptide
SQWNND

SEQ ID NO: 12 - protein, 5 aa, Artificial Sequence
  Description of Artificial Sequence Synthetic peptide
WDTAN

SEQ ID NO: 13 - protein, 5 aa, Artificial Sequence
  Description of Artificial Sequence Synthetic peptide
YEMAS

SEQ ID NO: 14 - protein, 15 aa, Artificial Sequence
  Description of Artificial Sequence Synthetic signal peptide
AAVALLPAVLLALLA

SEQ ID NO: 15 - protein, 14 aa, Artificial Sequence
  Description of Artificial Sequence Synthetic TAT-G peptide
GRKKRRQRRRPPQG

SEQ ID NO: 16 - protein, 8 aa, Artificial Sequence
  Description of Artificial Sequence Synthetic modified penetratin sequence Tr-pen
RRQKWKKG

SEQ ID NO: 17 - protein, 18 aa, Artificial Sequence
  Description of Artificial Sequence Synthetic penetratin
RQIKIWFQNRRMKWKKCC

(19) United States
(12) Patent Application Publication
(10) Pub. No.: US 2011/0105408 A1
(43) Pub. Date: May 5, 2011

(54) THERAPEUTIC PEPTIDES

(75) Inventor: Michael Valentine Agrez, Charlestown (AU)

(73) Assignee: INTER-K PTY LIMITED, Newcastle (AU)

(21) Appl. No.: 12/735,869

(22) PCT Filed: Feb. 23, 2009
(86) PCT No.: PCT/AU2009/000201

§ 371 Date: Nov. 15, 2010

(30) Foreign Application Priority Data
Feb. 22, 2008 (AU) 2008900848

(51) Int. Cl. A61K 38/00; C07K 14/00; A61P 35/00

(52) U.S. Cl. 514/19.3; 530/324; 530/350; 514/1.1; 514/19.5; 514/19.4

(57) ABSTRACT

The invention relates to dendrimer agents presenting polypetides providing the binding domain of a β integrin sub-unit for an ERK MAP kinase, or a variant or modified form of the binding domain, and the use of the dendrimers to inhibit growth of cancer cells. The peptides present more that 8 units of the polypeptide(s) examples of which include the peptide RSKAKNPLYR (SEQ ID No. 6).

### FIELD OF THE INVENTION

The invention relates to inhibition of the growth and/or proliferation of cancer cells, and dendrimer agents for same.

### BACKGROUND OF THE INVENTION

The spread of cancer cells involves tumour cell migration through the extracellular matrix scaffold, invasion of basement membranes, arrest of circulating tumour cells, and tumour cell extravasation and proliferation at metastatic sites. Detachment of cells from the primary tumour mass and modification of the peri-cellular environment aid penetration of tumour cells into blood and lymphatic vessels. It is the invasive and metastatic potential of tumour cells that ultimately dictates the fate of most patients suffering from malignant diseases. Hence, tumourigenesis can be viewed as a tissue remodelling process that reflects the ability of cancer cells to proliferate and digest surrounding matrix barriers. These events are thought to be regulated, at least in part, by cell adhesion molecules and matrix-degrading enzymes.

Cell adhesion receptors on the surface of cancer cells are involved in complex cell signalling which may regulate cell proliferation, migration, invasion and metastasis and several families of adhesion molecules that contribute to these events have now been identified including integrins, cadherins, the immunoglobulin superfamily, hyaluronate receptors, and mucins. In general, these cell surface molecules mediate both cell-cell and cell-matrix binding, the latter involving attachment of tumour cells to extracellular scaffolding molecules such as collagen, fibronectin and laminin.

Of all the families of cell adhesion molecules, the best-characterised is the family known as integrins. Integrins are involved in several fundamental processes including leucocyte recruitment, immune activation, thrombosis, wound healing, embryogenesis, virus internalisation and tumourigenesis. Integrins are transmembrane glycoproteins consisting of an alpha (α) and beta (β) chain in close association that provide a structural and functional bridge between extracellular matrix molecules and cytoskeletal components with the cell. The integrin family comprises 17 different α and 8β subunits, and the αβ combinations are subsumed under 3 subfamilies.

Excluding the leucocyte integrin subfamily that is designated by the β2 nomenclature, the remaining integrins are arranged into two major subgroups, designated β1 and αv based on sharing common chains.

In the β1 subfamily, the β1 chain combines with any one of nine α chain members (α1-9), and the a chain which associates with β1 determines the matrix-binding specificity of that receptor. For example, α2β1 binds collagen and laminin, α3β1 binds collagen, laminin and fibronectin, and α5β1 binds fibronectin. In the αv subfamily of receptors, the abundant and promiscuous αv chain combines with any one of five β chains, and a distinguishing feature of αv integrins is that they all recognise and bind with high affinity to arginine-glycine-aspartate (RGD) (SEQ ID. No. 1) sequences present in the matrix molecules to which they adhere.

The current picture of integrins is that the N-terminal domains of α and β subunits combine to form a ligand-binding head. This head, containing the cation binding domains, is connected by two stalks representing both subunits, to the membrane-spanning segments and thus to the two cytoplasmic domains. The β subunits all show considerable similarity at the amino acid level. All have a molecular mass between 90 and 110 kDa, with the exception of β4 which is larger at 210 kDa. Similarly, they all contain 56 conserved cysteine residues, except for β4 which has 48. These cysteines are arranged in four repeating patterns which are thought to be linked internally by disulphide bonds. The α-subunits have a molecular mass ranging from 150-200 kDa. They exhibit a lower degree of similarity than the β chains, although all contain seven repeating amino acid sequences interspaced with non-repeating domains.

The β subunit cytoplasmic domain is required for linking integrins to the cytoskeleton. In many cases, this linkage is reflected in localisation to focal contacts, which is believed to lead to the assembly of signalling complexes that include α-actinin, talin, and focal adhesion kinase (FAK). At least three different regions that are required for focal contact localisation of β1 integrins have been delineated (Reszka et al, 1992). These regions contain conserved sequences that are also found in the cytoplasmic domains of the β2, β3, β5, β6 and β7 integrin subunits. The functional differences between these cytoplasmic domains with regard to their signalling capacity have not yet been established.

The integrin β6 subunit was first identified in cultured epithelial cells as part of the αvβ6 heterodimer, and the αvβ6 complex was shown to bind fibronectin in an arginine-glycine-aspartate (RGD)-dependent manner in human pancreatic carcinoma cells (Sheppard et al, 1990). The β6 subunit is composed of 788 amino acids and shares 34-51% sequence homology with other integrin subunits β1-β5. The β6 subunit also contains 9 potential glycosylation sites on the extracellular domain (Sheppard et al, 1990). The cytoplasmic domain differs from other subunits in that it is composed of a 41 amino acid region that is highly conserved among integrin subunits, and a unique 11 amino acid carboxy-terminal extension. The 11 amino acid extension has been shown not to be necessary for localisation of β6 to focal contacts. In fact, its removal appears to increase receptor localisation. However, removal of any of the three conserved regions identified as important for the localisation of β1 integrins to focal contacts (Reszka et al, 1992) has been shown to eliminate recruitment of β6 to focal contacts (Cone et al, 1994).

The integrin αvβ6 has previously been shown to enhance growth of colon cancer cells in vitro and in vivo, and this growth-enhancing effect is due, at least in part, to αvβ6 mediated gelatinase B secretion (Agrez et al, 1999). What has made this epithelial-restricted integrin of particular interest in cancer is that it is either not expressed or expressed at very low levels on normal epithelial cells, but is highly expressed during wound healing and tumourigenesis, particularly at the invading edge of tumour cell islands (Breuss et al, 1995; Agrez et al, 1996).

Integrins can signal through the cell membrane in either direction. The extracellular binding activity of integrins can be regulated from the cell interior as, for example, by phosphorylation of integrin cytoplasmic domains (inside-out signalling), while the binding of the extracellular matrix (ECM) elicits signals that are transmitted into the cell (outside-in signalling). Outside-in signalling can be roughly divided into two descriptive categories. The first is ‘direct signalling’ in which ligation and clustering of integrins is the only extracellular stimulus. Thus, adhesion to ECM proteins can activate cytoplasmic tyrosine kinases (eg. focal adhesion kinase FAK) and serine/threonine kinases (such as those in the mitogen-activated protein kinase (MAPK) cascade) and stimulate lipid metabolism (eg. phosphatidylinositol-4,5-biphosphate (P₁ P₂) synthesis). The second category of integrin signalling is ‘collaborative signalling’, in which integrin-mediated cell adhesion modulates signalling events initiated through other types of receptors, particularly receptor tyrosine kinases that are activated by polypeptide growth factors. In all cases, however, integrin-mediated adhesion seems to be required for efficient transduction of signals into the cytosol or nucleus.

MAP kinases behave as a convergence point for diverse receptor-initiated signalling events at the plasma membrane. The core unit of MAP kinase pathways is a three-member protein kinase cascade in which MAP kinases are phosphorylated by MAP kinase kinases (MEKs) which are in turn phosphorylated by MAP kinase kinase kinases (e.g. Raf-1). Amongst the 12 member proteins of the MAP kinase family are the extracellular signal-regulated kinases (ERKs) (Boulton et al, 1991) activated by phosphorylation of tyrosine and threonine residues which is the type of activation common to all known MAP kinase isoforms. ERK 1/2 (44 kD and 42 kD MAPks, respectively) share 90% amino acid identity and are ubiquitous components of signal transduction pathways (Boulton et al, 1991). These serine/threonine kinases phosphorylate and modulate the function of many proteins with regulatory functions including other protein kinases (such as p90^{rsk}) cytoskeletal proteins (such as microtubule-associated phospholipase A₂), upstream regulators (such as the epidermal growth factor receptor and Ras exchange factor) and transcription factors (such as c-myc and Elk-1). ERKs play a major role in growth-promoting events, especially when the concentration of growth factors available to a cell is limited (Giancotti and Ruoslahti, 1999).

Recently, MAP kinases have been found to associate directly with the cytoplasmic domain of integrins, and the binding domains of β3, β5 and β6 for ERK2 have been characterised (see International Patent Application No. WO 2001/000677 and International Patent Application No. WO 2002/051993). Those patent applications also showed that growth of cancer cells can be inhibited by a peptide comprising the respective binding domains. The binding domain for β2 was also reported in International Patent Application No. WO 2005/037308.

The distribution of β6 integrin subunit within various tissues has been assessed by both in situ hybridisation and immunostaining and reported in the art. For instance, β6 mRNA in adult primate tissues was detected only in epithelial cells and at very low or undetectable levels in most normal tissues (Breuss et al, 1993). High-level expression of β6 has been observed in secretory endometrial glands while low-level expression was detected in the ductal epithelia of salivary gland, mammary gland and epididymis, in gall and urinary bladder, and in the digestive tract.

Immunostaining data have also shown that β6 expression is restricted to epithelia and is up-regulated in parallel with morphogenetic events, tumourigenesis and epithelial repair (Breuss et al, 1993; 1995). During development of the kidney, lung and skin, β6 is expressed by specific types of epithelial cells, whereas it is mostly undetectable in normal adult kidney, lung and skin. In contrast, high level expression of β6 has been observed in several types of carcinoma. For example, β6 is almost invariably neo-expressed in squamous cell carcinomas derived from the oral mucosa, and often focally localised at the infiltrating edges of tumour cell islands (Breuss et al, 1995). Moreover, expression of the β6 subunit has been observed in renal cell carcinoma and testicular tumour cell lines (Takiuchi et al, 1994) and 50% of lung cancers have been shown to express this subunit (Smythe et al, 1995).

Recent studies have also shown that αvβ6 is a major fibronectin-binding receptor in colorectal cancer (Agrez et al, 1996). In addition, normal colonic epithelium from cancer patients does not express αvβ6 in immunostaining studies, and as with squamous cell carcinomas from the oral mucosa (Thomas et al, 1997), maximal β6 expression in colon cancer has been observed at the invading edges of tumour cell islands (Agrez et al, 1996).

Indeed, the β6 subunit is widely observed in cancers of various origins (Breuss et al, 1995). For example, β6 is detected in at least 50% of bowel cancer tumours. Others have reported its presence in oropharyngeal cancers where it is also present and strongly expressed in the invading margins of the cancer cell islands as is commonly found in bowel cancer. In the oropharyngeal mucosa, no β6 is observed in the normal lining cells of the mouth but in both primary and metastatic tumours from the oropharyngeal mucosa, strong β6 expression is seen which does not correlate with degree of differentiation and in particular, is restricted to the basal layer of epithelial cells.

Expression of β6 is also up-regulated in migrating keratinocytes at the wound edge during experimental epidermal wound healing. αvβ6 is not expressed in normal epithelium (Jones et al, 1997). However, following experimental wounding, αv appears to switch its heterodimeric association from β5 to β6 subunit during re-epithelialisation. At day 3 after wounding, β6 is absent but then appears around the perimeter of the basal cells of the migrating epidermis.

In human mucosal wounds, maximal expression of β6 has been observed relatively late when epithelial sheets are fused and granulation tissue is present (Haapasalmi et al, 1996). Furthermore, those investigators observed maximal expression of tenascin with αvβ6 expression. Interestingly, freshly isolated keratinocytes have not been found to express β6 but begin to express this after subculturing. In contrast to persistent αvβ6 expression observed in colon cancer cells, new expression of αvβ6 in migrating keratinocytes is down-regulated to undetectable levels once re-epithelialisation is complete. Further, in normal unwounded skin, just as in other normal epithelia, αvβ6 expression is essentially absent.

Various methods have been employed in the past for enhancing the half-life of administered therapeutic peptides or for facilitating passage of such peptides into target cells in the past. Such methods have included coupling the peptide to a cell permeable moiety for facilitating passage into target cells. Such cell permeable moieties have included penetratin, signal peptides and fragments thereof, TAT derived peptides, dendrimers, and the like. Dendrimers comprise relatively large branched framework which provides scaffolding to which multiple copies of a peptide or other agent are coupled, and have been used to generate antibodies in vivo against epitopes defined by the peptide for subsequent targeting of cancer cells by the antibodies, or to generate an immune response to effect immunization of the individual.

Conventionally, the focus on the development of new therapeutics for the treatment of cancer is on the provision of small molecules, particularly when it is necessary for the therapeutic molecule to gain entry into the cytoplasm of cells via passage across the outer cell membrane to exert its effect. Typically also, it is desirable to avoid the generation of antibodies against the molecule, especially in the treatment of cancer which can involve prolonged and repeated periods of treatment with the molecule. This particularly applies to peptide therapeutics wherein the emphasis is on the identification of active shorter rather than longer peptides.

### SUMMARY OF THE INVENTION

The invention stems from the observation that administration of dendrimers presenting an anti-cancer polypeptide based on a binding domain of a β integrin subunit for an ERK MAP kinase, can provide enhanced inhibition of cancer cell growth compared to the polypeptide when coupled to at least some small molecule moieties for facilitating passage of the polypeptide into the cancer cells. It has also surprisingly been found that a dendrimer presenting 8 units of the polypeptide can increase basal, unstimulated ERK MAP kinase activity in the cancer cells (which is unwanted in the treatment of cancer) but that in one or more embodiments this can be avoided and indeed ERK MAP kinase activity inhibited in the cells at the same concentration of the dendrimer, when the dendrimer is modified to present more than 8 units of the same polypeptide. The reason why a dendrimer presenting 8 units of the polypeptide can activate ERK MAP kinase activity whereas ERK MAP kinase activity is inhibited when more than 8 units of the polypeptide is presented by the dendrimer is not known. Nevertheless, these startling findings provide a significant advance in the treatment of cancer.

In one aspect of the invention there is provided a dendrimer presenting more than 8 units of at least one polypeptide providing a cytoplasmic binding of a β integrin subunit for an ERK MAP kinase, or a variant or modified form thereof to which the MAP kinase binds.

In another aspect there is provided a pharmaceutical composition comprising a dendrimer embodied by the invention together with a pharmaceutically acceptable carrier.

In another aspect there is provided a method for inhibiting growth of a cancer cell, comprising treating the cell with an effective amount of a dendrimer presenting more than 8 units of at least one polypeptide providing a cytoplasmic binding of a β integrin subunit for an ERK MAP kinase, or a variant or modified form thereof to which the MAP kinase binds. The treatment of the cancer cell with the dendrimer can be in vitro or in vivo.

Hence, in another aspect of the present invention there is provided a method for prophylaxis or treatment of a cancer in a mammal, comprising administering to the mammal an effective amount of a dendrimer presenting more than 8 units of at least one polypeptide providing a cytoplasmic binding of a β integrin subunit for an ERK MAP kinase, or a variant or modified form thereof to which the MAP kinase binds.

In another aspect of the invention there is provided the use of a dendrimer presenting more than 8 units of at least one polypeptide providing a cytoplasmic binding of a β integrin subunit for an ERK MAP kinase, or a variant or modified form thereof to which the MAP kinase binds, for the prophylaxis or treatment of cancer in a mammal.

In another aspect of the invention there is provided the use of a dendrimer presenting more than 8 units at least one polypeptide providing a cytoplasmic binding of a β integrin subunit for an ERK MAP kinase, or a variant form thereof to which the MAP kinase binds, in the manufacture of a medicament for the prophylaxis or treatment of cancer in a mammal.

In at least some forms, the binding domain of the β integrin subunit incorporates an amino acid linker sequence that links opposite end regions of the binding domain together and which is not essential for the binding of the MAP kinase. One or more amino acids of the intervening amino acid sequence can be deleted or differ in the polypeptide compared to the binding domain of the β integrin subunit.

Typically, all of the amino acids in the intervening amino acid sequence will be deleted in the polypeptide compared to the binding domain.

Typically, the opposite end regions of the binding domain are defined by respective amino acid sequences, and the amino acid sequence identity of the opposite end regions remain unchanged in the polypeptide.

Typically also, the β integrin subunit is expressed by the cancer cells of the cancer. In other forms, the cancer cells essentially do not express the β integrin subunit.

In at least some embodiments, the polypeptide will comprise, or consist of, an amino acid sequence selected from the group consisiting of RSKAKWQTGTNPLYR (SEQ ID No: 2), RARAKWDTANNPLYK (SEQ ID No: 3), RSRARYEMASNPLYR (SEQ ID No: 4), KEKLKSQWNNDNPLFK (SEQ ID No: 5), RSKAKNPLYR (SEQ ID No: 6), RARAKNPLYK (SEQ ID No: 7), RSRARNPLYR (SEQ ID No: 8), and KEKLKNPLFK (SEQ ID No: 9).

The binding domain of the β integrin subunit (or a variant or modified form of the binding domain) can be incorporated in a fusion protein and the invention expressly extends to the use of such fusion proteins in dendrimers as described herein.

The β integrin subunit will normally be selected from the group consisting of β2, β3, β5, and β6. Most usually, the β integrin subunit will be β6.

Usually, the MAP kinase is ERK1 or ERK2 and most usually, is ERK2.

The dendrimer can be any dendrimer deemed suitable for use in a method embodied by the invention. The dendrimer can, for example, have branched organic framework to which the binding domain (or modified or variant form thereof) is coupled, such as framework formed by poly (amidoamine) (PAMAM), tris(ethylene amine) ammonia or poly (propylene imine) (Astramol™). In other forms, the dendrimer can have framework incorporating polyamino acids forming branching units to which the peptide is coupled. In at least some embodiments, the dendrimer has a framework of branching units formed by polyamino acids.

Typically, the dendrimer will have a plurality of layers/generations of polyamino acid branching units to which the peptide is coupled. The polyamino acid branching units are normally formed by lysine residues. The respective units of the peptide presented by the dendrimer can provide the same or different binding domains (or variant forms thereof) of β integrin subunits for ERK. Typically, the dendrimer will present monomers of the peptide(s). The dendrimer can also have a core from which the branching framework of the dendrimer extends.

The cancer can be selected from the group consisting of, but is not limited to, epithelial cell cancers, sarcomas, lymphomas and blood cell cancers, including leukemias such as myeloid leukemias, eosinophilic leukemias and granulocytic leukemias. For prophylaxis or treatment of a white blood cells cancer such as leukemia, the β subunit of the integrin may be β2 the expression of which is restricted to white blood cells (Hynes et al, 1992).

The mammal can be any mammal treatable with a method of the invention. For instance, the mammal may be a member of the bovine, porcine, ovine or equine families, a laboratory test animal such as a mouse, rabbit, guinea pig, a cat or dog, or a primate or human being. Typically, the mammal will be a human being.

Significant enhancement of the efficacy of the anti-cancer polypeptide may be obtained in one or more embodiments of the invention. Enhancement of the efficacy of the polypeptide provides the substantial advantage of enabling the dosage of the administered polypeptide to be likewise reduced, thereby potentially minimizing side effects associated with the treatment and/or providing an improved treatment outcome.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of this application.

The features and advantages of invention will become further apparent from the following detailed description of non-limiting embodiments.

### BRIEF DESCRIPTION OF THE ACCOMPANYING DRAWINGS

FIG. 1 is a schematic illustration of peptide dendrimer;

FIG. 2 (a) Shows a schematic illustration of a multiple antigen peptide dendrimer (MAP), incorporating eight peptide monomers. (b) An increase in the number of Lys branching units increases the number of surface amine groups;

FIG. 3 is a schematic illustration of a peptide dendrimer presenting 10 peptide monomers of the peptide RSKAKNPLYR (SEQ ID NO: 6) (referred to herein as dendrimer IK248B or Dend 10 10(4));

FIG. 4 is a graph showing inhibition of activated phospho-ERK in HT29 colon cancer cells exposed to the peptide dendrimer IK248 (5 μM for 1 hour) presenting 8 monomer units of the polypeptide RSKAKNPLYR (SEQ ID No. 6) (also referred to herein as Dend 8 or Dend 8 10(4));

FIG. 5 is a graph showing phospho-ERK levels in HT29 colon cancer cells treated with 20 μM IK248 dendrimer after 1 hour incubation with and without stimulation of the cells by foetal calf serum (FCS);

FIG. 6 is a graph showing inhibition of proliferation of HT29 colon cancer cells by the dendrimer IK248B presenting 10 monomer units of the peptide RSKAKNPLYR (SEQ ID No. 6);

FIG. 7 is a graph showing the effect of dendrimers Dend 9 10(4) and Dend 12 10(4) (presenting 9 and 12 monomers of the peptide RSKAKNPLYR (SEQ ID No. 6), respectively) on proliferation of HT29 human colon cancer cells cultured for 48 hours;

FIG. 8 is a graph showing the efficacy of the dendrimer IK248B (in which the peptide RSKAKNPLYR (SEQ ID No. 6) is bipegylated and comprised entirely of D-amino acids) in inhibiting proliferation of HT29 colon cancer cells compared to cisplatin, irinotecan (CPT-11) and 5-fluorouracil (5FU);

FIG. 9 is a graph showing treatment of HT29 colon cancer cells with peptide dendrimer presenting 8 monomer units of the peptide RARAKNPLYK (SEQ ID No. 7) (Dend8 β3) (solid triangles) or 8 monomers of peptide RSRARNPLYR (SEQ ID No. 8) (Dend8 β5) (solid squares);

FIG. 10 is a graph showing inhibition of HT29 colon cancer tumour growth in a BALB/c mouse model by the dendrimer IK248B (solid squares) compared to a vehicle only control (solid diamonds); and

FIG. 11 is a graph showing in vivo stability of the IK248B dendrimer (in which the peptide RSKAKNPLYR (SEQ ID No. 4) is bipegylated and comprised entirely of D-amino acids) in a non-tumour bearing mouse model where mouse serum has been extracted at 5, 10, 30 and 60 minute time points following intravenous injection of peptide into the mouse, and the serum then tested for its ability to inhibit growth of HT29 colon cancer cells in vitro.

### DETAILED DESCRIPTION OF EMBODIMENTS OF THE INVENTION

The polypeptide presented by a dendrimer as described herein may provide the binding domain of the β-integrin subunit for the ERK MAP kinase, or vary from the binding domain by one or more amino acids. The polypeptide may also, or alternatively, differ by one or more amino acids from one or both regions of the β-integrin subunit which flank the binding domain.

By the term “binding domain” is meant the minimum length of contiguous amino acid sequence of the β-integrin subunit required for binding of the ERK MAP kinase substantially without compromising the optimum level of binding with the MAP kinase. Moroever, the term “binding domain” includes those binding domains encoded by naturally occurring mutant and polymorphic alleles.

By the term “variant form” of the binding domain of the β integrin subunit for the ERK MAP kinase is meant an amino acid sequence that differs from the binding domain by one or more amino acids essentially without adversely effecting binding by the MAP kinase.

By the term “modified form” is meant an amino acid sequence in which the binding domain of the β integrin subunit for the ERK MAP kinase has been modified by one or more amino acid changes essentially without adversely effecting the binding by the MAP kinase.

Variant and modified forms of the binding domain include derivatives and peptidomimetics of the binding domain. A variant or modified form of the binding domain will generally include 2 or more positively charged amino acid residuess (each independently positively or negatively charged) and typically, a minimum of 3 positively charged amino acids (e.g., H is, Lys, and/or Arg).

Typically, the binding domain will have opposite end regions that are linked together by a number of intervening amino acids (i.e., an amino acid linker sequence) which are not essential for binding of the MAP kinase to the β-integrin subunit and can be deleted.

The provision of a polypeptide useful in a dendrimer as described herein (e.g., a modified form of the binding domain of the β integrin subunit incorporating the binding domain) can be achieved by the addition, deletion and/or the substitution of one or more amino acids of the binding domain with another amino acid or amino acids.

Inversion of amino acids and any other mutational change that results in alteration of an amino acid sequence are also encompassed. For example, one or more amino acids of the non-essential intervening amino acid linker sequence of the binding domain can be deleted or substituted for another amino acid or amino acids, (e.g., conservative amino acid substitution(s)). Such modified polypeptides can be prepared by introducing nucleotide changes in a nucleic acid sequence such that the desired amino acid changes are achieved upon expression of the mutagenised nucleic acid sequence, or for instance by synthesising an amino acid sequence incorporating the desired amino acid changes, which possibilities are well within the capability of the skilled addressee.

Further, a modified binding domain or polypeptide as described herein can incorporate an amino acid or amino acids not encoded by the genetic code, or amino acid analog(s). For example, D-amino acids rather than L-amino acids can be utilised. Indeed, a peptide useful in an embodiment of the invention may consist partly or entirely of D amino acids. D-peptides can be produced by chemical synthesis using techniques that are well-known in the art. Accordingly, in some embodiments, the peptide(s) may include L-amino acids, D-amino acids or a mixture of L- and D-amino acids. The synthesis of peptides including D-amino acids can inhibit peptidase activity (e.g., endopeptidase) as is known in the art, and thereby enhance stability and increase the half-life of the peptide in vivo compared to the corresponding L-peptide.

Likewise, the N-terminal or C-terminal ends of the polypeptides/peptides can be modified to protect against or inhibit in vivo degradation (e.g., by endopeptidases). For instance, the C-terminus of the polypeptides can be amidated to protect against endopeptidase degradation. Alternatively, the N- or C-terminal end of a polypeptide as described herein can also be pegylated to render it less resistant to degradation by proteases in vivo or to inhibit their clearance from the circulation via the kidneys. Methods for pegylation of polypeptides/peptides are well known in the art and all such methods are expressly encompassed. Typically, a pegylated polypeptide used in a method embodied by the invention will be coupled to 2 or more monomer units of polyethylene glycol (PEG) and generally, from about 2 to about 11 monomers of PEG (i.e., (PEG)ₙ where n equals from 2 to 11). Most usually, n will be 2.

Substitution of an amino acid may involve a conservative or non-conservative amino acid substitution. By conservative amino acid substitution is meant replacing an amino acid residue with another amino acid having similar stereochemical properties (e.g., structure, charge, acidity or basicity characteristics) and which does not substantially adversely effect the binding activity of the binding domain. For example, a polar amino acid may be substituted with another polar amino acid, conservative amino acids changes being well known to the skilled addressee.

The sequence identity between amino acid sequences as described herein can be determined by comparing amino acids at each position in the sequences when the sequences are optimally aligned for the purpose of comparison. The sequences are considered the same at a position if the amino acids at that position are the same amino acid residue. Alignment of sequences can be performed using any suitable program or algorithm such as for instance, by the Needleman and Wunsch algorithm (Needleman and Wunsch, 1970). Computer assisted sequence alignment can be conveniently performed using standard software programs such as GAP which is part of the Wisconsin Package Version 10.1 (Genetics Computer Group, Madison, Wis., United States) using the default scoring matrix with a gap creation penalty of 50 and a gap extension penalty of 3.

Typically, a polypeptide useful in a dendrimer as described herein will have an overall amino acid sequence identity with the β integrin subunit of at least about 40% and more usually, at least about 50%, 60%, or 70% or greater and most preferably, at least about 80%, 90% or 95% sequence identity or greater. The sequence identity with the binding domain of the β integrin subunit may be greater than the overall amino acid sequence identity between the two sequences, and will usually be greater than about 60%, 70% or 80%, and more usually will be about 90%, or 95% or greater. However, it will be understood that the overall sequence identity of the polypeptide, or the sequence identity of the polypeptide with the binding domain, can be any specific value or range within the particular values specified above. For instance, the amino acid sequence identity of the polypeptide may be at least 66% or 75% or greater, and all such sequence identities and ranges are expressly encompassed by the invention.

A derivative of a polypeptide useful in a method embodied by the invention may be provided by cleavage cyclisation and/or coupling of the parent molecule with one or more additional moieties that improve solubility, lipophilic characteristics to enhance uptake by cells, stability or biological half-life, decreased cellular toxicity, or for instance to act as a label for subsequent detection or the like. A derivative may also result from post-translational or post-synthesis modification such as the attachment of carbohydrate moieties or chemical reaction(s) resulting in structural modification(s) such as the alkylation or acetylation of amino acid residues or other changes involving the formation of chemical bonds.

The term “polypeptide” is used interchangeably herein with peptide. For instance, it will be understood that peptide agents such as RSKAKNPLYR (SEQ ID No: 6) and KEKLKNPLFK (SEQ ID No: 9) fall within the scope of the term polypeptide.

Peptide dendrimers are particularly suitable for use in methods of the invention. Peptide dendrimers in at least some embodiments of the invention present units of the polypeptide inibitor coupled to a branched framework of polyamino acids (typically lysine branching units). The dendrimer will typically have at least 3 layers/generations of amino acid branching units, the units of the polypeptide inhibitor being coupled to the outermost layer/generation of the amino acid branching units such that the dendrimer presents more than 8 units of the polypeptide. While momoner units of the polypeptide are preferred, in other embodiments, dendrimers incorporating multiple units of the polypeptide (multimers) (e.g., (RSKAKNPLYR)n, wherein n is the number of repeats of the polypeptide (typically 1-3)) coupled to polyamino acid branching units of the dendrimer may be utilized. Hence, the units of the polypeptide presented by the dendrimer can be monomer units, multimer units and/or mixtures of monomer and multimer units of the polypeptide.

The anti-cancer polypeptide can be bonded to the outermost layer/generation of polyamino acid branching units forming the framework of the dendrimer, or be synthetically assembled on the polyamino acid branching units of the dendrimer. More particularly, the synthesis of dendrimers useful in one or more methods embodied by the invention can be achieved by divergent or convergent synthesis strategies.

The divergent strategy is a direct approach by which the dendrimer is built stepwise in a continuous operation on a solid support through solid-phase synthesis. Stepwise synthesis involves synthesis of the branching core of the dendrimer followed by synthesis of the polypeptide inhibitor in a continous manner. The divergent strategy is particularly suitable for the synthesis of dendrimers with a framework of a trifunctional acid (eg., polyamino acid). Such solid phase synthesis schemes are the method of choice for the synthesis of lysine branching units where di-protected lysine is used to produce a branching framework of multiple levels of lysines. The diamino nature of lysine results in each additional level of lysine effectively doubling the number of sites upon which the polypeptide inhibitor may be synthesized directly.

The convergent strategy is an indirect, modular approach by which the polypeptide and branching core unit are prepared separately and then coupled together. Core units with branching framework used in the convergent synthesis of dendrimers are commercially available, and are typically formed from organic amino compounds such as poly (amidoamine) (PAMAM), tris(ethylene amine) ammonia or poly (propylene imine) (Astramol™) to which separately prepared inhibitor is normally covalently linked.

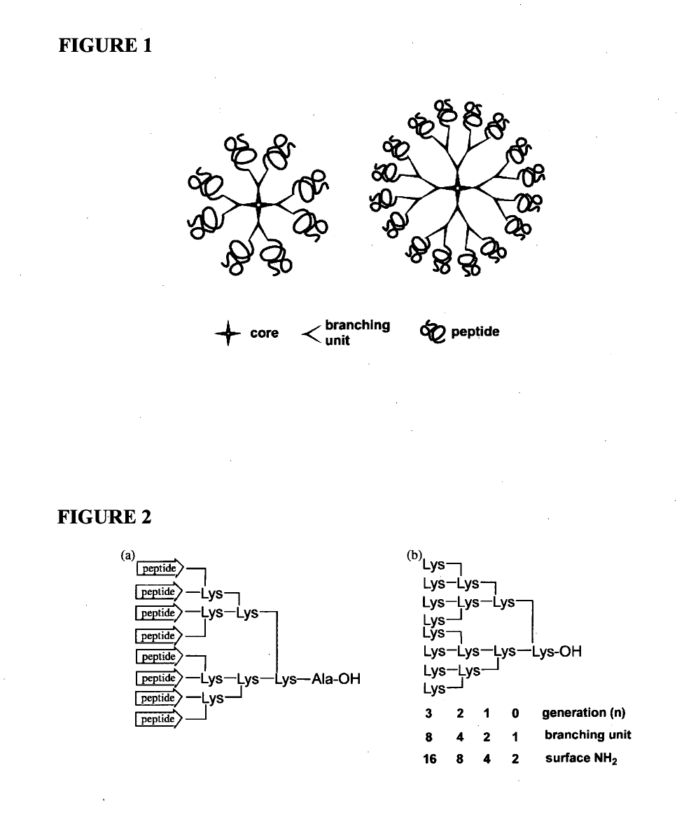

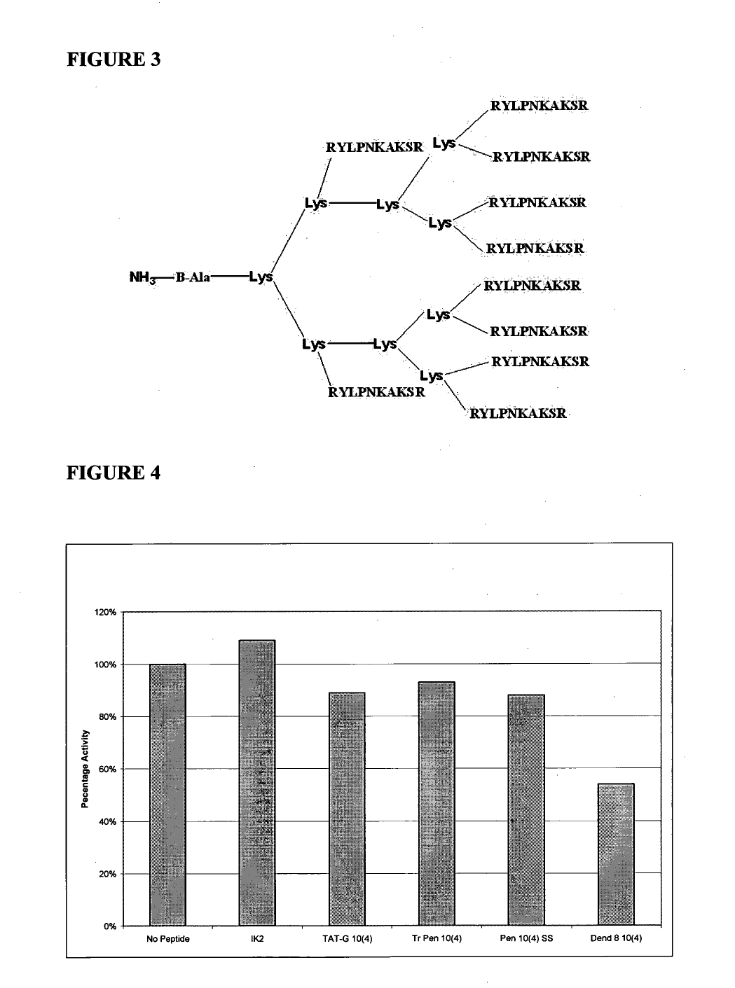

Suitable peptide dendrimer framework to which an inhibitor as described herein can be coupled, and methods for the provision of peptide dendrimers, are for example described in Lee et al, 2005; Sadler and Tam, 2002; and Cloninger, 2002, the entire contents of which are incorporated herein in their entirety by reference. Examples of peptide dendrimers of the type suitable for use in embodiments of the present invention are schematically illustrated in FIG. 1. and FIG. 2. (Sadler, K., and Tam, J. P., 2002), and in FIG. 3.

A dendrimer used in a method embodied by the invention presents more than 8 units of the polypeptide (e.g., 9, 10 or 12 units). While the polypeptides presented by the dendrimer will normally all be the same, mixtures of polypeptides as described herein may also be used. For example, half of the units of the polypeptide presented by the dendrimer can provide the binding domain of the β6 integrin subunit for the ERK MAP kinase while the remaining units of the polypeptide present the binding domain of the β5 integrin subunit (or variant or modified forms of these binding domains). However, it will be understood that the ratio of the different anti-cancer polypeptide agents may also be varied in a dendrimer. The peptide(s) presented by a dendrimer used in a method embodied by the invention will also typically be N- or C-terminal protected against proteolytic degradation (e.g., by amidation, pegylation (i.e., the addition of PEG units) or the like). Methods such as pegylation of polypeptides are within the scope of the skilled addressee, and all such methods are expressly encompassed.

Typically, the polypeptide presented in the dendrimer in accordance with embodiments of the invention will have a length of about 60 amino acids or less. Usually, the polypeptide will have a length of more than 5 amino acids and will normally, be up to about 50 amino acids, 40, 35, 30, 25, 20 or 15 amino acids in length. In at least some embodiments, the polypeptide may have a length in a range of from 6, 7, 8, 9 or 10 amino acids up to about 14, 15, 16, 17, 18, 19, 20, or 25 amino acids. However, it will be understood that polypeptides of all specific lengths and length ranges within those identified above that are suitable for use in a dendrimer as described herein are expressly encompassed (e.g., 13 or 14 amino acids or from 10 to 22 amino acids etc.).

The binding domain of the β integrin subunits β2, β3, β5 and β6 for the MAP kinase ERK2 are described in International Patent Application No. WO 2001/000677, and International Patent Application No. 2002/051993. The binding domain of the β2 integrin subunit for ERK2 is described in International Patent Application No. 2005/037308. The disclosures of all of these international patent applications are expressly incorporated herein by reference in their entirety. In particular, further polypeptide agents for inhibiting the binding of a β integrin subunit to a MAP kinase and which are suitable for being incorporated into a dendrimer as described herein are also described in those applications, as well as methodology for the localisation and characterization of the binding domains.

More particularly, the binding domain may be localised by assessing the capacity of respective overlapping peptide fragments of the cytoplasmic binding domain of a β integrin subunit for the ERK MAP kinase. The specific amino acid sequence which constitutes the binding domain may then be determined utilising progressively smaller peptide fragments. For this purpose, test peptides are readily synthesised to a desired length involving deletion of an amino acid or amino acids from one or both of the N-terminal and C-terminal ends of the larger peptide fragment(s), and tested for their ability to bind with the ERK MAP kinase. This process is repeated until the minimum length peptide capable of binding with the ERK MAP kinase substantially without compromising the optimum observed level of binding is identified.

The identification of amino acids that play an essential role in the ERK MAP kinase—β integrin interaction may be achieved with the use of further synthesised test peptides in which one or more amino acids of the sequence are deleted or substituted with a different amino acid or amino acids to determine the effect on the binding ability of the peptide. Typically, substitution mutagenesis will involve substitution of selected ones of the amino acid sequence with alanine or other neutrally charged amino acid.

Nucleotide and amino acid sequence data for the β6 integrin subunit for example is found in Sheppard et al, 1990. ERK1 and ERK2 have high overall amino acid sequence identity, with ERK1 having about 96% sequence identity to a 26 mer amino acid sequence of ERK2 providing the binding site for β6 (see International Patent Application No. WO 2002/051993. The nucleotide and amino acid sequence for ERK2 is for instance found in Boulton et al, 1991. Reference to such published data allows the ready design of polypeptides useful in the dendrimers described herein and the provision of the corresponding nucleic acid sequences encoding the polypeptides.

In order to constrain a polypeptide or other agent in a three dimensional conformation required for binding, it may be synthesised with side chain structures or incorporating cysteine residues which form a disulfide bridge. A polypeptide or other agent may also be cyclised to provide enhanced rigidity and thereby stability in vivo, and various such methods are known in the art

As described above, a polypeptide useful in a dendrimer embodied by the invention can comprise, or consist of, the binding domain of the β integrin subunit, or a variant or modified form thereof in which one or more amino acids of the intervening amino acid sequence of the binding domain that are not essential for binding of the MAP kinase are deleted. As an example, the binding domain of β6 comprises the amino acid sequence RSKAKWQTGTNPLYR (SEQ ID No: 2). However, the intervening amino acid sequence WQTGT (SEQ ID No: 10) is not essential for binding of the MAP kinase ERK2. That is, even if the sequence WQTGT (SEQ ID No: 10) is deleted, a peptide with the amino acid sequence RSKAKNPLYR (SEQ ID No: 6) is still bound by ERK2. Similarly, the binding domains of β2, β3 and β5 for ERK2 are provided by KEKLKSQWNNDNPLFK (SEQ ID No. 5), RARAKWDTANNPLYK (SEQ ID No: 3) and RSRARYEMASNPLYR (SEQ ID No: 4), respectively. Deletion of the intervening sequences SQWNND (SEQ ID No. 11), WDTAN (SEQ ID No: 12) and YEMAS (SEQ ID No: 15) from these sequences yields the 10 mer peptides KEKLKNPLFK (SEQ ID No. 9), RARAKNPLYK (SEQ ID No: 7) and RSRARNPLYR (SEQ ID No: 8), all of which still bind to ERK2. As will be appreciated, the peptide RSKAKNPLYR (SEQ ID No. 6) has 80% sequence identity with peptide RSRARNPLYR (SEQ ID No: 8) and vice versa.

Alignment of the binding domains off β2, β3 and β5 and β6 results in the concensus scheme R/K×R/K×R/K−xxxxx NPL Y/F R/K wherein R/K is either arginine or lysine, Y/F is either tyrosine or phenylalanine, x may be any amino acid, and “−” (i.e., the dash) is an amino acid that is not essential and can be deleted, and a polypeptide utilized in a dendrimer as described herein may be represented by, or comprise, this consensus scheme. The amino acid designated by “−” can be a serine residue or may be another amino acid such as threonine, tyrosine, asparagine or glutamine. Typically, the polypeptide has an amino acid sequence represented by R/K×R/K*R/K−xx*x*NPL Y/F R/K wherein each * is independently a hydrophobic amino acid or an amino acid selected from the group consisting of serine, tyrosine and threonine. Hydrophobic amino acids are non-polar amino acids and examples include alanine, valine, leucine, isoleucine, and phenylalanine. Hence, the entire intervening amino acid sequence indicated by −xxxxx (or one or more of the amino acids of that sequence) may also be deleted such that the polypeptide comprises, or consists of, the sequence R/K×R/K×R/K NPL Y/F R/K.

The use of fusion proteins incorporating a polypeptide providing the binding domain of a β integrin subunit for a MAP kinase is expressly encompassed by the invention. Polypeptides and fusion proteins or the like may be synthesised or produced using conventional recombinant techniques. Nucleic acid encoding a fusion protein may for instance be provided by joining separate DNA fragments encoding peptides or polypeptides having the desired amino acid sequence(s) by employing blunt-ended termini and oligonucleotide linkers, digestion to provide staggered termini as appropriate, and ligation of cohesive ends. Alternatively, PCR amplification of DNA fragments can be utilised employing primers which give rise to amplicons with complementary termini which can be subsequently ligated together (eg. see Ausubel et al. (1994) Current Protocols in Molecular Biology, USA, Vol. 1 and 2, John Wiley & Sons, 1992; Sambrook et al (1998) Molecular cloning: A Laboratory Manual, Second Ed., Cold Spring Harbour Laboratory Press, New York). Polypeptides and fusion proteins may be expressed in vitro and purified from cell culture for administration to a subject, or cells may be transfected with nucleic acid encoding a polypeptide or fusion protein for in vitro or in vivo expression thereof. The nucleic acid will typically first be introduced into a cloning vector and amplified in host cells, prior to the nucleic acid being excised and incorporated into a suitable expression vector for transfection of cells. Methods for the cloning, expression and purification of polypeptides useful in dendrimers of the invention are also well within the scope of the skilled addressee.

Liposomes, ghost bacterial cells, synthetic polymer agents, ultracentrifuged nanoparticles and other anucleate nanoparticles (e.g., produced as a result of inactivating the genes that control normal bacterial cell division (De Boer P. A., 1989) may be loaded with dendrimers or peptides as describede herein and used for targeted delivery of the dendrimers to cancer cells (e.g., via bispecific antibodies carried/coated on the nanoparticles). Such nanoparticles may be formulated for injection, or for oral consumption for passage through the acid environment of the stomach for release and uptake of the dendrimer via the small intestine.

The toxicity profile of a dendrimer as described herein may be tested on normal and abnormal cells such as cancer cells by evaluation of cell morphology, trypan-blue exclusion, assessment of apoptosis and cell proliferation studies (e.g., cell counts, ³H-thymidine uptake and MTT assay).

The cancer treated by a method of the invention may for instance be selected from the group consisting of leukaemias, myeloid leukaemias, eosinophilic leukaemias, granulocytic leukaemias, and cancer of the liver, tongue, salivary glands, gums, floor and other areas of the mouth, oropharynx, nasopharynx, hypopharynx and other oral cavities, oesophagus, gastrointestinal tract, stomach, small intestine, duodenum, colon, rectum, gallbladder, pancreas, larynx, trachea, bronchus, lung, breast, uterus, cervix, ovary, vagina, vulva, prostate, testes, penis, bladder, kidney, thyroid, and skin. Typically, the cancer will be an epithelium cancer and most usually, a non-dermal cancer.

The dendrimer(s) will typically be formulated into a pharmaceutical composition comprising a pharmaceutically acceptable carrier and/or excipient for administration to the intended subject. The dendrimer, or a pharmaceutical composition comprising the dendrimer can, though not exclusively, be administered orally, intravenously, parenterally, rectally, subcutaneously, by infusion, topically such as in the treatment of skin cancers, intramuscularly, intraperitonealy, intranasally and by any other route deemed appropriate. The pharmaceutical composition can for example be in the form of a liquid, suspension, emulsion, syrup, cream, ingestable tablet, capsule, pill, suppository, powder, troche, elixir, or other form that is appropriate for the selected route of administration.

In particular, pharmaceutical compositions embodied by the invention include aqueous solutions. Injectable compositions will be fluid to the extent that syringability exists and typically, will normally stable for a predetermined period to provide for storage after manufacture. Moreover, pharmaceutically acceptable carriers include any suitable conventionally known solvents, dispersion media, physiological saline and isotonic preparations or solutions, and surfactants. Suitable dispersion media can for example contain one or more of ethanol, polyols (e.g., glycerol, propylene glycol, liquid polyethylene glycol and the like), vegetable oils and mixtures thereof.

For oral administration, any orally acceptable carrier can be used. In particular, the dendrimer may be formulated with an inert diluent, an assimilable edible carrier or it may be enclosed in a hard or soft shell gelatin capsule.

Topically acceptable carriers conventionally used for forming creams, lotions or ointments for internal or external application can be employed. Such compositions can be applied directly to a site to be treated or via by dressings and the like impregnated with the composition.

A pharmaceutical composition as described herein can also incorporate one or more preservatives suitable for in vivo and/or topical administration such as parabens, chlorobutanol, phenol, sorbic acid, and thimersal. In addition, prolonged absorption of the composition may be brought about by the use in the compositions of agents for delaying absorption such as aluminium monosterate and gelatin. Tablets, troches, pills, capsules and the like containing the dendrimer can also contain one or more of the following: a binder such as gum tragacanth, acacia, corn starch or gelatin; a disintegrating agent such as corn starch, potato starch or alginic acid; a lubricant such as magnesium sterate; a sweetening agent such as sucrose, lactose or saccharin; and a flavouring agent.

The use of ingredients and media as described above in pharmaceutical compositions is well known. Except insofar as any conventional media or ingredient is incompatible with the dendrimer, use thereof in therapeutic and prophylactic compositions as described herein is included.

It is particularly preferred to formulate parenteral compositions in dosage unit form for ease of administration and uniformity of dosage. Dosage unit form as used herein is to be taken to mean physically discrete units suited as unitary dosages for the subject to be treated, each unit containing a predetermined quantity of active agent calculated to produce the desired therapeutic or prophylactic effect in association with the relevant carrier used. When the dosage unit form is for example, a capsule, tablet or pill, various ingredients may be used as coatings (e.g., shellac, sugars or both) to otherwise modify the physical form of the dosage unit or to facilitate administration to the individual.

A pharmaceutical composition will generally contain at least about 1% by weight of the dendrimer. The percentage may of course be varied and may conveniently be between about 5% to about 80% w/w of the composition or preparation. As will be understood, the amount of the dendrimer in the composition will be such that a suitable effective dosage will be delivered to the subject taking into account the proposed route of administration. Preferred oral compositions embodied by the invention will contain between about 0.1 μg and 15 g of the dendrimer.

The dosage of the dendrimer will depend on a number of factors including whether the dendrimer is to be administered for prophylactic or therapeutic use, the condition for which the dendrimer is intended to be administered, the severity of the condition, the age of the subject, and related factors including weight and general health of the individual as may be determined by the physician or attendant in accordance with accepted principles. For instance, a low dosage may initially be given which is subsequently increased at each administration following evaluation of the individual's response. Similarly, the frequency of administration may be determined in the same way that is, by continuously monitoring the individual's response between each dosage and if necessary, increasing the frequency of administration or alternatively, reducing the frequency of administration.

Typically, the dendrimer will be administered in accordance with a method of the invention to provide a dosage of the polypeptide inhibitor of up to about 50 mg/kg body weight of the individual and preferably in a range of from about 20 mg/kg to 40 mg/kg body weight. In at least some embodiments, the dendrimer will be administered to provide a dosage of the polypeptide in a range of from about 5 to 25 mg/kg body weight, usually in a range of from about 5 mg/kg to about 20 mg/kg and more usually, in a range of from 10 mg/kg to about 20 mg/kg. When administered orally, up to about 20 g of the dendrimer may be administered per day, (e.g., 4 oral doses each comprising 5 g of the dendrimer).

With respect to intravenous routes, particularly suitable routes are via injection into blood vessels which supply a tumour or particular organs to be treated. Agents may also be delivered into cavities such for example the pleural or peritoneal cavity, or be injected directly into tumour tissue. Suitable pharmaceutically acceptable carriers and formulations useful in compositions of the present invention may for instance be found in handbooks and texts well known to the skilled addressee, such as “Remington: The Science and Practice of Pharmacy (Mack Publishing Co., 1995)”, the contents of which is incorporated herein in its entirety by reference.

The present invention will be described herein after with reference to a number of non-limiting Examples.

### Example 1

### Materials and methods

### 1.1 Cell Lines and Culture

HT29 (colorectal adenocarcinoma), DU145 (prostate carcinoma), MCF-7 (breast adenocarcinoma) cells were obtained from the American Type Culture Collection (ATCC, Manassas, Va., United States). MKN45 (gastric carcinoma) were obtained from the Cancer Research Laboratory, University of New South Wales, Sydney, Australia. All cell lines were maintained in DMEM medium (Invitrogen, Carlsbad, Calif., United Sattes) containing 10% v/v filtered foetal calf serum (FCS; Invitrogen) and 20 mM Hepes (ThermoTrace, Melbourne, Vic, Australia). Cell lines were maintained at 37° C. in a humid atmosphere containing 5% CO₂. Cells were passaged at pre-confluent densities using a solution containing 0.05% trypsin and 0.5 mM EDTA (Invitrogen).

### 1.2 Cell Proliferation (MTT) Assay

Single cell suspensions of viable trypsinised cells were seeded into 96-well tissue culture plates at a density of 2×10³ cells per well in a volume of 100 μl of a suitable culture media with foetal calf serum (FCS) (e.g., Dulbecco's Modified Eagles Medium (DMEM), with 10% v/v FCS, glutamine, Hepes, and antibiotics). A set of triplicate wells was prepared for each concentration of the peptide dendrimer being tested. Additional sample wells containing untreated cells or media alone were set up in each treatment plate and processed in parallel as reference controls. A zero-time plate of untreated cells and media-alone wells was simultaneously prepared and an MTT assay carried out on this plate at the time of test peptide dendrimer addition to treatment plates. All plates were cultured for 24 hours before addition of the test dendrimer. To prepare the MTT solution 100 mg of MTT (3-(4,5-dimethylthiazolyl-2)-2,5-diphenyltetrazolium bromide) (Cat #. M-128, Sigma, St Louis Mo.), is mixed with 20 ml of PBS at pH 7.4. The resulting solution is filter sterilized (0.2 μM syringe filter) and stored at 4° C. protected from light until use. MTT substrate is cleaved in growing cells to yield a water insoluble salt. After solubilisation of the salt crystals, a coloured product is produced the measurement of which allows quantitation of the proliferative activity of the cultured cells.

Appropriate concentrations of the test dendrimer were prepared by dilution of freshly prepared sterile 1 mM stock solutions into cell culture media to give a final well volume of 200 μl containing 10% v/v FCS. The zero-plate was processed by addition of MTT at this time. Cell culture was continued for a further 24 or 48 hours before addition of 20 μl of MTT in PBS (5 mg/ml, 0.2 um filter sterilised). The MTT cell proliferation assay measures cell proliferation rate and, in instances where cell viability is compromised, the assay indicates a comparative reduction in cell viability. After a 3 hour incubation in the presence of MTT (5% CO₂ in air at 37° C.), plates were centifuged at 450 g for 5 minutes, supernatant removed by gentle suction and precipitated tetrazolium salt resuspended into 150 μl DMSO:glycine (0.1 M glycine, 0.1 M NaCl pH 10.5) (6:1 v/v) solution. Plates were gently vortexed to complete solubilisation of crystalline material and absorbance was read at 540-550 nm using a microplate reader. Sample data were processed to determine the comparative growth of treated samples relative to untreated controls.

### 1.3 Determination of Phospho-ERK and Total-ERK ELISA

HT29 cells were harvested using 0.5% trypsin EDTA (Invitrogen) and 5000 cells were resuspended into 200 μL, and added to each well of a clear NUNC tissue culture treated 96 well plate (NUNC). Cells were seeded in DMEM media (Invitrogen) supplemented with 10% v/v foetal calf serum (FCS, Invitrogen), 1% L-Glutamine (Invitrogen) and 2% 1M HEPES buffer solution (Invitrogen). Cells were then incubated overnight at 37° C. The following day, the growth medium was replaced with 100 μL of serum-free DMEM supplemented with 1% L-glutamine and 2% 1M HEPES buffer solution, and incubated for a further 24 hours at 37° C. Dendrimers were added to test wells in serum free medium to provide final concentrations of the dendrimers ranging from 0.1-20 μLA. The total volume of each well was 200 uL, and cells were incubated at 37° C. for times ranging from 5 minutes to 4 hours. In those studies in which cells were subjected to stimulation by serum, 22 μL FCS (10% v/v final concentration) was added to wells for the final ten minutes of the culture period at 37° C. Serum-free media (SFM) only (22 μL) was added to non-serum stimulated cells.

Phospho-ERK levels were determined using the FACE ERK1/2 ELISA kit according to the manufacturer's instructions (Active Motif, kit obtained from Australian Biosearch, WA, Australia). Briefly, media was replaced and cells fixed with 4% formaldehyde in PBS, and after a 1 hour with antibody blocking buffer (supplied), the primary phospho-ERK or total-ERK antibody was added and incubated overnight at 4° C. Antibody dilution buffer only was added to control test wells containing no primary antibody. The next day, HRP-conjugated secondary antibody was added for one hour before plates were developed and the absorbance measured at 450 nm using a Labsystems Multiskan EX microplate reader (Labsystems, Thermo Labsystems, UK).

### 1.4 Peptide Dendrimers

Peptide dendrimers of the type illustrated in FIG. 3 comprising lysine branching units coupled to monomer units of either a polypeptide inhibitor of the binding of ERK2 to the β integrin subunits β3, β5 or β6 were employed in the following Examples.

### Example 2

### Peptide Dendrimer Comprising 4 or 8 Monomer Units of the Polypeptide RSKAKNPLYR (SEQ ID No. 6) Inhibits the Proliferation of HT29 Colon Cancer Cells

Peptide dendrimers comprising 4 (Dend 4) or 8 (referred to herein as dendrimer IK248, Dend 8 or Dend 8 10(4)) monomer units of the polypeptide RSKAKNPLYR (SEQ ID No. 6) were found to inhibit proliferation of HT29 colon cancer cells as assessed by the MTT assay described in Example 1.2. Notably, IK248 was found to be substantially more effective at inhibiting cell growth/proliferation than the peptide dendrimer comprising 4 monomers of the polypeptide (24 hour incubation period).

In another study, the IK248 dendrimer also inhibited growth/proliferation of MKN45 gastric carcinoma cells, MCF-7 breast cancer cells and DU145 prostate cancer cells. In this study, the cells were incubated in the presence of the dendrimer for 48 hours.

### Example 3

### Phospho-ERK Levels in HT29 Human Colon Cancer Cells Treated with Various Agents

The effectiveness of the peptide dendrimer IK248 described in Example 2 in inhibiting growth factor mediated activation of ERK in HT29 cancer cells (i.e., (FCS stimulated) compared to various agents comprising the polypeptide RSKAKNPLYR (SEQ ID No. 6) coupled to different peptide facilitator moieties for facilitating passage of the polypeptide across the plasma cell membrane into the cytosol of the cells was evaluated. Phospho-ERK levels in the cells following treatment with 5 μM IK248 (Dend 8) for 1 hour or polypeptide-facilitator moiety agents (each at 5 μM for the same duration) were measured essentially as described in Example 1.3. The facilitator moieties utilised were the signal peptide fragment AAVALLPAVLLALLA (SEQ ID No. 16), the TAT-G peptide GRKKRRQRRRPPQG (SEQ ID No. 17), a modified pentratin sequence Tr-Pen RRQKWKKG (SEQ ID No. 18), and the penetratin peptide RQIKIWFQNRRMKWKKC_{S—S}C (SEQ ID No. 19) wherein S—S indicates a disulphide bridge between the adjacent cysteine residues.

The percentage inhibition of activated phospho-ERK by IK248 and the polypeptide-facilitator moieties in the HT29 cells at the 1 hour time point is shown in FIG. 4. As can be seen, IK248 exhibited at least 30% greater inhibition than the test agent which displayed the closest level of inhibition, namely the TAT-G RSKAKNPLYR (SEQ ID No. 6) polypeptide. At 4 hours, 5 μM IK248 exhibited approx. 95% inhibition of activated phospho-ERK compared to relatively low level inhibition by the polypeptide-facilitator moieties (data not shown).

### Example 4

### Phospho-Erk Levels in HT29 Human Colon Adenocaricnoma Cells Treated with Peptide Dendrimers Presenting Peptide RSKAKNPLYR (SEQ ID No. 6)

### 4.1 Cell Culture and Treatment Conditions

The peptide RSKAKNPLYR (SEQ ID. No. 6) or peptide dendrimers of the type illustrated in FIG. 3 comprising lysine branching units presenting either 8 (IK248) or 10 (IK248B) monomer units of the peptide RSKAKNPLYR (SEQ ID No. 6) were utilised in this study. Phospho-ERK levels were evaluated by ELISA utilising an Active Motif FACE ERK1/2 ELISA kit (Australian Biosearch, WA, Australia) as described in Example 1.3.

### 4.2 Results

4.2.1 Phospho-ERK Levels in Absence of Growth Factors (HT29 Cells not Stimulated with HI FCS)

In dose response studies, the level of phospho-ERK in HT29 cells treated with lower concentrations of the IK248 dendrimer (1 μM-10 μM with 4 hr incubation) were found to incease relative to control cells thereby indicating activation of basal, unstimulated ERK by the dendrimer. Moreover, HT29 cells incubated with 5 μM IK248 dendrimer showed markedly increased phospho-ERK levels at all time points evaluated (5, 10 and 30 minutes, and 1 hour) compared to control cells.

In contrast, dendrimer IK248B (presenting 10 monomer units of peptide RSKAKNPLYR (SEQ ID No. 6)) did not stimulate basal phospho-ERK levels in HT29 cells, with levels of phopho-ERK in HT29 cells remaining below that of control cells for all doses of the dendrimer tested (0.1, 0.5, 1 and 5 μM dose responsewith 4 hr incubation).

4.2.2 Phospho-ERK Levels in Presence of Growth Factors (HT29 Cells Stimulated with HI FCS)

Following stimulation with HI FCS, phospho-ERK levels in HT29 control cells were observed to increase around 3 fold. In cells treated with 20 μM IK248 (1 hour incubation), phospho-ERK levels were lower than in non-FCS stimulated cells treated with the dendrimer, but remained higher than phospho-ERK basal levels in control cells not treated with the dendrimer (see FIG. 2) or stimulated with FCS. Total ERK levels remained essentially unaffected in HT29 cells treated with the IK248 dendrimer.

In HT29 cells treated with dendrimer IK248B, phospho-ERK levels decreased relative to control cells with progressively increasing dosages of the IK248B dendrimer, and were almost totally abrogated at a dose of 5 μM. ERK activity (as indicated by phospho-ERK levels) was entirely inhibited in HT29 cells by IK248B at a concentration of 10 μM (data not shown).

Further, phospho-ERK levels in HT29 cells treated with the IK248B dendrimer were significantly reduced compared to normal skin fibroblast cells indicating substantial selectivity of the dendrimer for cancer cells compared to normal cells (data not shown). In a further study, a dendrimer of the same type but presenting 10 monomer units of a scrambled form of the RSKAKNPLYR (SEQ ID No. 6) peptide was relatively ineffective at inhibiting ERK activation in FCS stimulated HT29 colon cancer cells compared to the IK248B dendrimer presenting the unscrambled RSKAKNPLYR (SEQ ID No. 6) peptide.

### Example 5

### Inhibition of Proliferation in HT29 Human Adenocarcinoma Cells

HT29 cells were cultured for 48 hours in the presence of selected dendrimers and proliferation of the cells was assessed by MTT assay essentially as described in Example 1.2. The results were calculated as percentage proliferation of control cells (not treated with dendrimer).

### 5.1 Inhibition of Proliferation by Dendrimer IK248B

HT29 cells were treated with peptide dendrimer IK248B and the results are shown in FIG. 4. As can be seen, proliferation of the cells was inhibited by the dendrimer.

### 5.2 Dendrimer Size

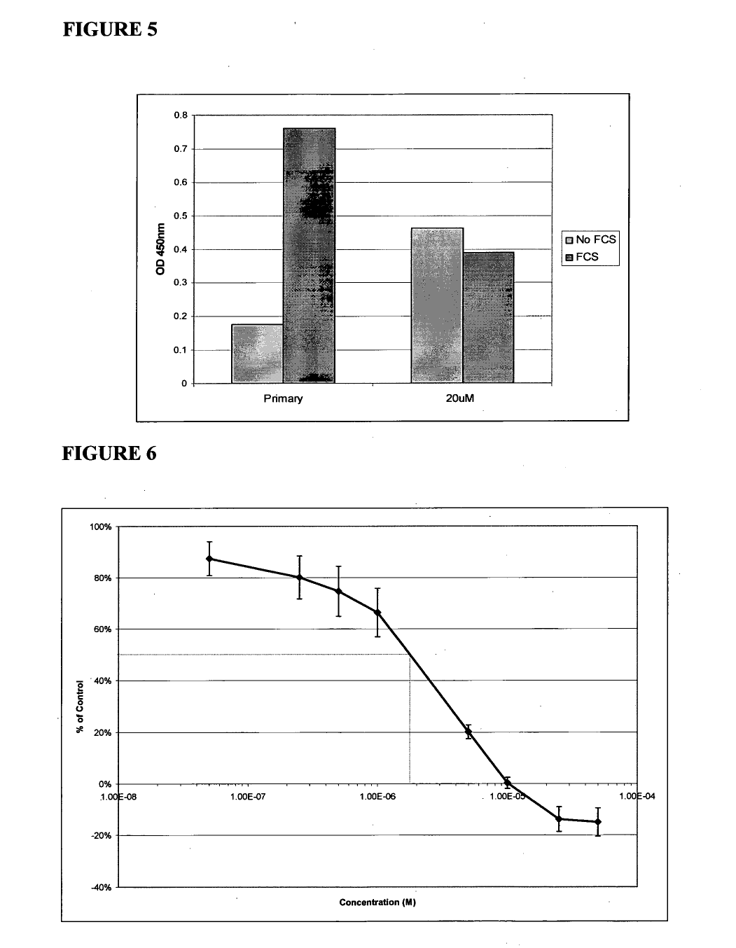

Dendrimers of the type described in Scheme 3 above presenting 9 (Dend 9 (10)4) or 12 (Dend 12 10(4)) monomer units of the peptide RSKAKNPLYR (SEQ ID No. 6) were assessed for capacity to inhibit proliferation of the HT29 cells. As shown in FIG. 5, Dend 12 was more effective than Dend 9 in inhibiting proliferation of the cells. When compared to dendrimer IK248B (presenting 10 monomer units of peptide RSKAKNPLYR (SEQ ID No. 6)) Dend 12 10(4) showed a small improvement in IC₅₀ value (1 μM versus 1.8 μM) but no increase in the dendrimer concentration required for total kill (namely 10 μM for both Dend 12 (10)4 and IK248B) was obtained. Dendrimer IK248B was in turn more effective than dendrimer IK248 (presenting 8 monomer units of the RSKAKNPLYR peptide (SEQ ID No. 6)) (IC₅₀'s of 1.8 μM and 5 μM, respectively).

### 5.3 Use of Peptide RSKAKNPLYR (SEQ ID No. 6) Composed of D Amino Acids

The efficacy of the peptide dendrimer 11(248B in which the monomer units of the RSKAKNPLYR peptide (SEQ ID No. 6) were composed entirely of D amino acids and pegylated with two polyethylene glycol (PEG) units at their carboxy terminal end (identified as modified (Mod.) 11(248B or Peg2 Dend10 D-10(4)) in inhibiting proliferation of human HT29 cells was compared to cisplatin, irinonectin (CPT-11) and 5-fluorouracil (5FU). Proliferation of the cells was assessed by MTT assay and the results are shown in FIG. 6. As can be seen, the modified 11(248B dendrimer effected substantially greater inhibition of proliferation of the cells than cisplatin, CPT-11 and 5FU alone.

### Example 6

### Treatment of HT29 Colon Cancer Cells with Peptide Dendrimers Presenting RARAKNPLYK (SEQ ID No. 7) or RSRARNPLYR (SEQ ID No. 8)

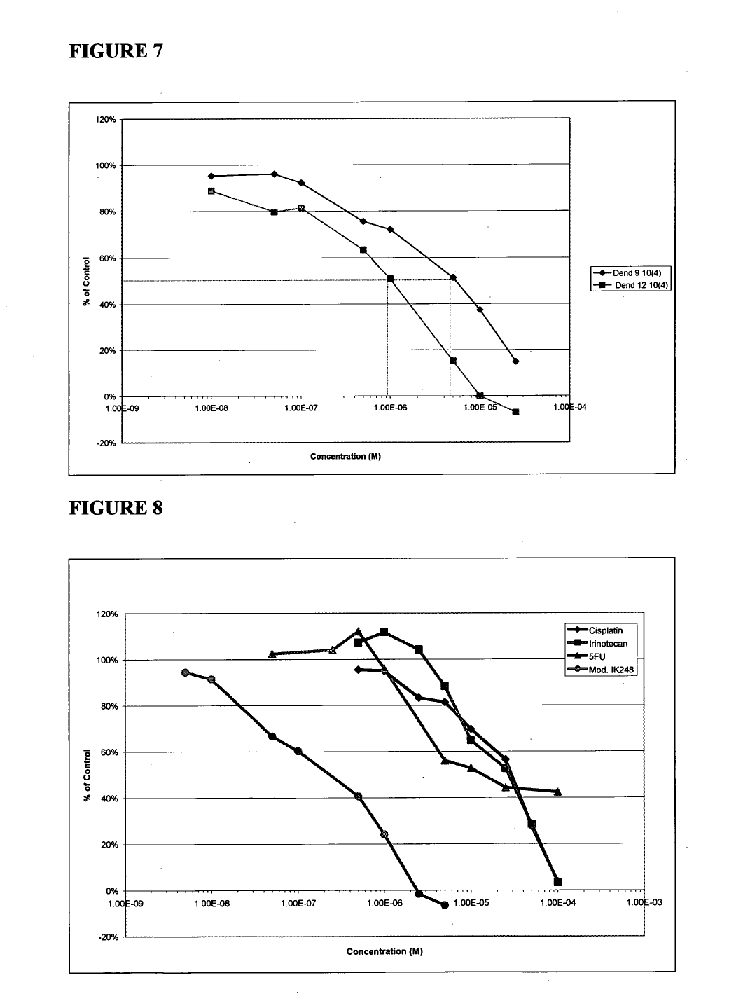

HT29 colon cancer cells were treated with peptide dendrimers of the type illustrated in FIG. 3 presenting 8 monomer units of the 10 mer 133 based peptide (RARAKNPLYK (SEQ ID No. 7)) (Dend8 133) or the 135 based peptide (RSRARNPLYR) (SEQ ID No. 8) (identified as Dend8 135). Test cells were exposed to the dendrimers for 48 hours and proliferation of the cells was evaluated using the MTT assay essentially as described above in Example 1.2. Absorbance was read at 550 nm using a microtitre plate reader, and the percentage inhibition of proliferation of the test cells was calculated relative to untreated control cells. The results are shown in FIG. 7. As can be seen, both of dendrimers Dend8 β3 and Dend8 β5 inhibited proliferation of the HT29 cancer cells, although Dend8 β5 was more effective.

### Example 7

### Inhibition of Tumour Growth in a Mouse Model

### 7.1 Materials

Reagents for the culture of HT-29 human colorectal adenocarcinoma cells were obtained from the following suppliers: RPMI 1640 cell culture medium, FBS and HBSS from Invitrogen Australia (Mt Waverley, VIC, Australia); penicillin-streptomycin, phosphate buffered saline (PBS) and trypan blue from Sigma-Aldrich (Castle Hill, NSW, Australia).

### 7.2 Tumour Cell Production

HT29 human colorectal adenocarcinoma cells were cultured in RPMI 1640 cell culture medium supplemented with 10% v/v FCS and 50 IU/mL penicillin-streptomycin. The cells were harvested by trypsinisation, washed twice in HBSS and counted. The cells were then resuspended in HBSS to a final concentration of 2×10⁷ cells/mL.

### 7.3 Test System

Species: Mouse (Mus musculus)
Strain: BALB/c nu/nu
Source: University of Adelaide (Waite Campus, Urrbrae, SA, Australia)
Total number of animals in study: 20 females
Number of study groups: 2 (1 test, 1 control)
Number of mice per group: 10
Body weight range: 20.61-25.27 g at onset of treatment (mean 22.27 g)
Age range: 10-12 weeks at onset of treatment.

### 7.4 Tumour Inoculation

Prior to inoculation the skin on the injection site (dorsal right flank) was swabbed with alcohol. The needle was introduced through the skin into the subcutaneous space just below the animal's right shoulder, and 100 μL of cells (2×10⁶ cells) were discharged. The treatment of mice began nine days after HT29 cell inoculation, the average tumour volume was 68 mm³ (average variability of 6.1%).

### 7.5 Body Weight and Tumour Measurements

Body weight and tumour dimensions (length and diameter) were measured for all animals on the first day of treatment (day 0) and then three times per week, including the termination day of the study (Day 24).

### 7.6 Administration

Mice were randomized, based on body weight, into two groups of ten mice on Day 0 of the study. The peptide dendrimer IK248B presenting 10 monomer units of the peptide RSKAKNPLYR (SEQ ID No. 6) (see Example 8) was used in this study. The vehicle control (phosphate buffered saline (PBS)) and IK248B dendrimer (20 mg/kg) were each administered by intra-tumoural injection once daily for five consecutive days, beginning on Day 0.

The vehicle control and IK248B dendrimer were administered at a dosing volume of 4.762 mL/kg (100 μL) based on a 21 g mouse. Each animal's body weight was measured immediately prior to dosing. The volume of dosing solution administered to each mouse was calculated and adjusted based on individual body weight.

### 7.7 Sample Collection and Calculations

Tumours were excised from all mice post mortem and weighed. Tumour volume was calculated using the equation:

V(mm³)=length×diameter²×p/6

Tumour variability was calculated using the equation:

Variability(%)=(SEM_{Tumour volume}/Tumour volume_{Average})×100

ΔT/ΔC(%) was calculated using the following equation:

ΔT/ΔC(%)=(Δvolume_{Treatment}/ΔVolume_{Control})×100

where Δ=change in volume from day 0 to the final measurement day or nominated day of interest.

### 7.8 Statistical Calculations

All statistical calculations were performed using SigmaStat 3.0 (SPSS Australasia, North Sydney, NSW, Australia). A two-sample t-test was used to determine the significance in body weight change within a treatment group between day 0 and day 4, and between day 0 and the termination day of the study. A One-Way Analysis of Variance (ANOVA) was performed on tumour volumes measured in all mice at the end of the study. Where significant differences were found in the data, the All Pairwise Multiple Comparison Procedures (Holm-Sidak Method) were performed. A two-sample t-test was used to show a significant difference between the tumour weight data for the vehicle control and Dend 10 10(4) treatment groups. A p value of less than 0.05 was considered significant.

### 7.9 Results

TABLE 3
Tumour volume analysis

| Treatment | | Days Post-Initial Treatment | | | | | | | | | |
|---|---|---|---|---|---|---|---|---|---|---|---|
| Group | Parameter | 0 | 2 | 4 | 7 | 11 | 14 | 16 | 18 | 21 | 24 |
| Vehicle | Average | 68.1 | 119.2 | 209.3 | 333.2 | 469.7 | 641.6 | 782.2 | 937.0 | 1143.2 | 1372.6 |
| | Stdev | 18.5 | 82.8 | 57.8 | 107.5 | 173.3 | 237.2 | 241.5 | 278.1 | 286.1 | 372.5 |
| | Sem | 5.9 | 9.1 | 18.3 | 34.0 | 54.8 | 75.0 | 76.4 | 87.9 | 90.5 | 117.8 |
| | Delta avg | 0.0 | 51.1 | 141.2 | 265.1 | 401.6 | 573.4 | 714.1 | 868.8 | 1075.1 | 1304.5 |
| | dT/dC [%] | 100.0 | 100.0 | 100.0 | 100.0 | 100.0 | 100.0 | 100.0 | 100.0 | 100.0 | 100.0 |
| Dend 10- | Average | 68.0 | 136.6 | 211.9 | 248.6 | 300.9 | 323.9 | 391.0 | 476.9 | 601.5 | 702.1 |
| 10(4) | Stdev | 19.8 | 62.6 | 123.7 | 132.1 | 110.8 | 177.5 | 207.2 | 245.7 | 280.9 | 351.9 |
| | Sem | 6.3 | 19.8 | 39.1 | 41.8 | 35.0 | 56.1 | 65.5 | 77.7 | 88.8 | 111.3 |
| | Delta avg | 0.0 | 68.6 | 144.0 | 180.6 | 232.9 | 255.9 | 323.1 | 408.9 | 598.7 | 710.4 |
| | dT/dC [%] | 100.0 | 134.3 | 102.0 | 68.1 | 58.0 | 44.6 | 45.2 | 47.1 | 55.7 | 54.5 |

As shown more clearly in FIG. 8, tumour growth was markedly inhibited by the dendrimer IK248B (Dend10 10(4)) (solid squares) compared to the vehicle only control (solid diamonds). In particular, the growth of the tumours slowed noticeably between Day 5 to Day 15 in the treatment groups relative to the control group. The average tumour weight in the control group at the end of the study was 0.962 g±0.124 SEM compared to 0.437 g±0.072 SEM for the 11(248B treatment group, a highly significant outcome (P≦0.003).

### Example 8

### Stability of Dendrimer In Vivo

### 8.1 Materials

96 well cell culture plates (Becton-Dickinson, North Ryde, NSW, Australia). CellTiter-Blue® (Alamar Blue™) Cell Viability Assay (Promega, Madison, Wis., USA). RPMI 1640 cell culture medium, FBS and HBSS (Invitrogen Australia, Mt Waverley, VIC, Australia). Penicillin-streptomycin and Trypan Blue (Sigma-Aldrich, Castle Hill, NSW, Australia). Saline (Baxter Australia, NSW, Australia). Spectramax Gemini XPS Fluorometer (Adelab Scientific, Adelaide, SA, Australia). Mouse serum (MS) was collected from BALB/c mice by orbital bleeding followed by centrifugation.

### 8.2 Animal Model

| | |
|---|---|
| Species: | Mouse (Mus musculus) |
| Strain: | BALB/c |
| Source: | University of Adelaide (Waite |
| | Campus, Urrbrae, SA, Australia) |
| Total number of animals in study: | 9 (for in vivo study) |
| Body weight range: | 17.8-20.7 g at onset of treatment |
| | (mean 19.12 g) |
| Age range at start: | 8-10 weeks at onset of treatment |

All animals received a detailed physical examination, including body weight measurement. The animals were found to be in satisfactory health and were housed in a single micro-isolator cage. The cage was clearly labelled with a cage card indicating study number, gender, and prescribed dose concentration and dose volume. Each animal was identified by a transponder (Bar Code Data Systems, Botany Bay, NSW, Australia) that could be scanned with a barcode reader (DataMars LabMax I). The transponder was implanted by subcutaneous injection between the shoulder blades while the mouse was under isofluorane-induced anaesthesia.

The animals were kept in a controlled environment (targeted ranges: temperature 21±3° C., humidity 30-70%, 10-15 air changes per hour), with a light/dark cycle each of 12 hours, and under barrier (quarantine) conditions. Temperature and relative humidity were monitored continuously. All animals were subjected to the same environmental conditions. Any deviations from the targeted ranges were judged not to have affected the well-being of the animals. A standard certified commercial rodent diet (Rat and Mouse Cubes, Speciality Feeds, Glen Forrest, WA, Australia) and tap water were provided to the animals ad libitum. Food supply was sterilised by autoclaving, and water supply was sterilised by acidification with hydrochloric acid (pH 2.4-2.7). The animals were allowed to become accustomed to the laboratory environment for 24 hours before the study commenced.

### 8.3 Cell Culture

The human colorectal adenocarcinoma cell line HT-29 was sourced from the American Type Culture Collection (ATCC) (Rockville, Md., USA). The HT-29 cells were cultured in RPMI 1640 cell culture medium, supplemented with 10% v/v FCS and 50 IU/mL penicillin-streptomycin. All cells were grown at 37° C. in humidified cell culture incubators supplied with 95% air/5% CO₂. The cells used in this study were used after passage 2.

Cells were harvested by trypsinisation, washed twice in HBSS and counted. The cells were then re-suspended in the appropriate culture medium to a concentration of 1×10⁴ cells/mL. 50 μL of this dilution (containing 4,000 cells) were added to each of the wells of 96 well plates.

### 8.4 Dendrimer Peg2 Dend10 D-10(4)

| | |
|---|---|
| Description: | White powder |
| Molecular weight: | 14,843 daltons |
| Lot No.: | T40800 |
| Expiry Date: | N/A |
| Storage conditions: | 4° C. |
| Handling precautions: | Standard laboratory precautions |

The Peg2 Dend10 D-10(4) dendrimer was supplied in 1 mg vials containing 0.72 mg of pure peptide. On the dosing day, 4 vials were reconstituted with 226 μL of sterile phosphate buffered saline (PBS). The contents of the 4 vials were pooled together before administration. For in vitro studies, this stock of Peg2 Dend10 D-10(4) was used as a positive control and was diluted in cell culture medium to give final concentrations of 0.5 μL and 1.0 The mouse serum used for the positive control was prepared from untreated mice (Group 1).

Peg2 Dend10 D-10(4) dendrimer was administered a single 15 mg/kg dose, i.v. via the tail vein, in a dosing volume of 4.7 mL/kg. Each animal's body weight was measured immediately prior to dosing. The volume of dosing solution administered to each mouse was calculated and adjusted based on individual body weight.

### 8.5 Sample Collection and Termination

The 9 mice used in this study were allocated into 3 groups of 3. Group 1 mice were left untreated and had blood drawn. Group 2 mice were bled 5 and 30 minutes after Peg2 Dend10 D-10(4) treatment and Group 3 animals were bled 15 and 60 minutes after Peg2 Dend10 D-10(4) treatment. In all cases, blood was drawn via orbital bleed. Whole blood samples were collected into heparinised capillary tubes, transferred into fresh tubes and placed on ice. The samples were centrifuged (1000×g) for 2 to 3 minutes at 4° C. The plasma component was collected into fresh tubes and transferred for in vitro testing at 4° C. All mice were euthanised by terminal bleed via cardiac puncture, under isofluorane-induced anaesthesia.

### 8.6 Cell Proliferation Assays

HT-29 cells were plated in 2×96-well plates, one plate was assayed using the CellTiter-Blue® reagent 24 hours post-seeding (6 wells) and the second plate incubated for a further 72 hours in the presence of the Peg2 Dend10 D-10(4) dendrimer in 20% mouse sera and then subjected to CellTiter-Blue® assay. The average value obtained after 24 hours growth was subtracted from values obtained for the 96 hours incubation (72 hours in the presence of the dendrimer). The percentage of inhibition was calculated using the formula:

Inhibition(%)=[1−(Fluorescence at a given dose/Fluorescence of untreated cells)]×100

### 8.7 Celltitre-Blue® Assay

Following incubation of cells, 20 μL of CellTiter-Blue® was added to each well and incubated at 37° C. in a cell culture incubator. Fluorescence was measured using a Spectramax Gemini XPS Fluorometer.

### 8.8 Results

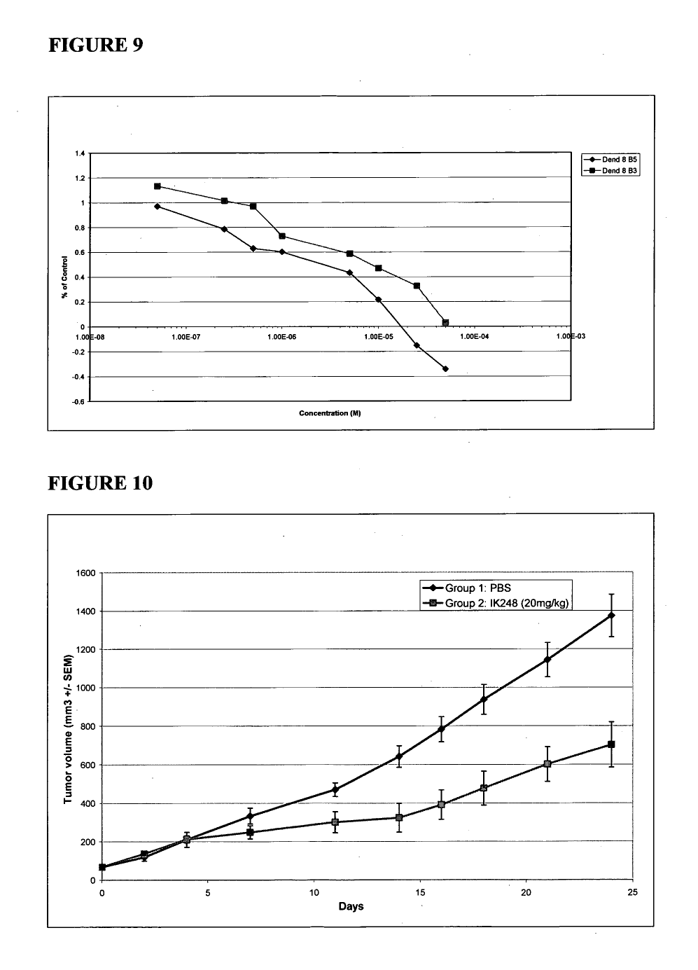

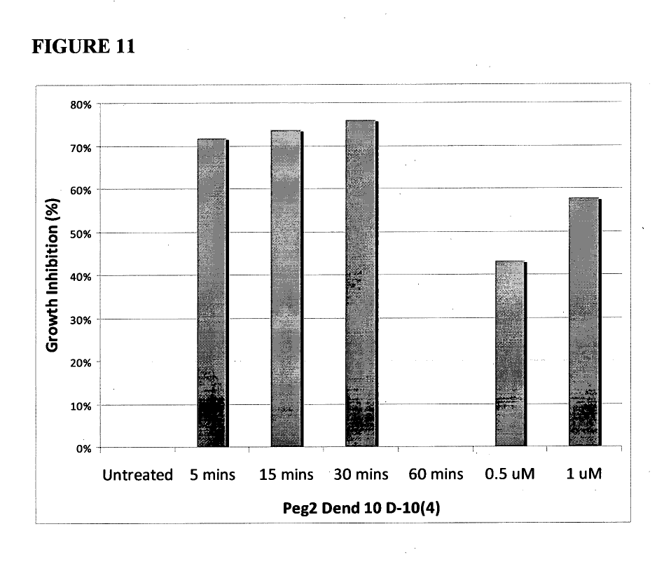

Results are shown in FIG. 9. In the in vitro study, greater than 40% inhibition of HT29 cancer cell proliferation was observed for the dendrimer at a concentration of 0.5 μM and approx. 58% inhibition was observed at a concentration of the dendrimer at 1 μM.

Pharmacokinetic studies were performed in non-tumour-bearing Balb/c mice. Serum from the mice was obtained at various time intervals after intravenous administration of the dendrimer. The presence of active peptide (RSKAKNPLYR, SEQ ID No. 6) in mouse serum was determined ex vivo by assessing the ability of 20% mouse serum to inhibit proliferation of HT29 cells in vitro.

In vitro growth inhibition of the HT29 cells in the serum of over 70% for the 5, 15 and 30 minute time points was observed. Inhibition at 60 minutes was almost zero. The 60 minute time point represents high variability between the three animals injected with the dendrimer, with one mouse giving a very high reading and arguably being an outlier. If the results for that animal are ignored, the inhibition at the 60 minute time point would be approx. 67%.

The concentration of the peptide RSKAKNPLYR (SEQ ID No 6) in peripheral mouse blood at the time of injection was approx. 10.6 μM (0.315 mg in 2 mL of total mouse blood). These results suggest that the concentration of the Peg2 Dend 10 D-10(4) dendrimer in the peripheral blood at 5.15 and 30 minutes after IV administration is at least higher than 1 μM.

These data clearly show that the Peg2 Dend 10 D-10(4) dendrimer is present in peripheral mouse blood for at least 30 minutes after IV administration.

Although a number of preferred embodiments have been described, it will be appreciated by persons skilled in the art that numerous variations and/or modifications may be made without departing from the spirit or scope of the invention as broadly described. The present embodiments are, therefore, to be considered in all respects as illustrative and not restrictive.

### LITERATURE REFERENCES

1. Agrez, M. V., Bates R. C., Mitchell, D., Wilson, N., Ferguson, N., Anseline, P. and Sheppard. D., Multiplicity of fibronectin-binding αv integrin receptors in colorectal cancer. Br. J. Cancer 73, 887-892 (1996).
2. Agrez, M. V., Gu, X., Turton, J., Meldrum, C., Niu, J., Antalis, T. and Howard, E. W., The βvβ6 integrin induces gelatinase B secretion in colon cancer cells. Int. J. Cancer 81, 90-97 (1999).
3. Boulton, T. G., Nye, S. H. and Robbins, D. J., ERKs: a family of protein-serine/threonine kinases that are activated and tyrosine phosphorylated in response to insulin and NGF. Cell 65, 663-675 (1991).
4. Breuss, J. M., Gallo, J., De Lisser, H. M., Klimanskaya, I. V., Folkesson, H. G., Pittet, J. F., Nishimura, S. L., Aldape, K., Landers, D. V., Carrenter, W., Gillet, N., Sheppard, D., Mathay, M., Albeda, S. M., Kramer, R. H., and Pytela, R., Expression of the β6 integrin in development, neoplasia and tissue repair suggests a role in epithelial remodelling. J. Cell Sci. 108, 2241-2251 (1995).
5. Breuss, J. M., Gillet, N., Lu, L., Sheppard, D. and Pytela, R., Restricted distribution of integrin beta 6 mRNA in primate epithelial tissues. J. Histochem. Cytochem. 41, 1521-1527 (1993).
6. Cloninger, M. J. Biological applications of dendrimers. Curr. Opin. Chem. Biology. 6, 742-748 (2002)
7. Cone, R. I., Weinacker, A., Chen, A. and Sheppard, D., Effects of beta subunit cytoplasmic domain deletions on the recruitment of the integrin alpha v beta 6 to focal contacts. Cell Adhes. Comm. 2, 101-113 (1994).
8. De Boer P A, Crossley, R E, and Rothfield, L I. A division inhibitor and topological specific factor coded for by the miicell locus determine proper placement of the division septum in E. coli. Cell, 56; 641-649, 1989.
9. Giancotti, F. G. and Ruoslahti, E., Integrin signalling. Science 285, 1028-1032 (1999).
10. Haapasalmi, K., Zhang, K., Tonneson, M., Olerud, J., Sheppard, D., Salo, T., Kramer, R., Clark, R. A. F., Uitlo, V-J. and Larjava, H., Keratinocytes in human wounds express avb6 integrin. J. Invest. Dermatol. 106, 42-48 (1996).
11. Howe, A., Aplin, A. E., Alahari, S. K. and Juliano, R. L., Integrin signaling and cell growth control. Curr. Opin. Cell Biol. 10, 220-231 (1998).
12. Hynes, R. O., Integrins: versatility, modulation and signaling in cell adhesion. Cell 69, 11-25 (1992).
13. Jones, J., Watt, F. M. and Speight, P. M., Changes in the expression of alpha v integrins in oral squamous cell carcinomas. J. Oral Path. & Med. 26, 63-68 (1997).
14. Lee, C. C., MacKay, J. A., Frechet, J. M. and Szoka, F. C. Designing dendrimers for biological applications. Nature Biotech. 23, 1517-1526 (2005).
15. Needlemen, S. B., and Wunsch, C. C. A general method applicable to the search for similarities in the amino acid sequence of two proteins. J. Mol. Biol. 48(3), 443-53, 1970.
16. Reszka, A. A., Hayashi, Y. and Horwitz, A. F., Identification of amino acid sequences in the integrin beta 1 cytoplasmic domain implicated in cytoskeletal association. J. Cell Biol. 117, 1321-1330 (1992).
17. Sadler, K. and Tam, J. P. Peptide dendrimers: applications and synthesis. Rev. Mol. Biotechnology. 90, 195-229, (2002).
18. Sheppard, D., Rozzo, C., Starr, L., Quaranta, V., Erle, D. J. and Pytela, R., Complete amino acid sequence of a novel integrin β subunit (β6) identified in epithelial cells using the polymerase chain reaction. J. Biol. Chem. 265, 11502-11507 (1990).
19. Smythe, W. R., Lebel, E., Bavaria, J. E., Kaiser, L. R. and Albelda, S. M., Integrin expression in non-small cell carcinoma of the lung. Cancer & Metastasis Reviews 14, 229-239 (1995).
20. Takiuchi, H., Kanokogi, M., Fujimoto, N., Hanafusa, T., Kyo, M., Ichikawa, Y., Nagano, S., Fulcunishi, T., Yabumoto, H. and Ihara, H., Expression of integrin molecule in urological tumour cell lines by using RT-PCR method. Jap. J. Urology 85, 584-588 (1994).
21. Thomas, G. J., Jones, J. and Speight, P. M., Integrins and oral cancer. Oral Oncology 33, 381-388 (1997).

1. A dendrimer presenting more than 8 units of at least one polypeptide providing a cytoplasmic binding of a β integrin subunit for an ERK MAP kinase, or a variant or modified form thereof to which the MAP kinase binds.

2. The dendrimer according to claim 1 wherein the polypeptide comprises or consists of the binding domain of the β integrin subunit.

3. The dendrimer according to claim 1 wherein the polypeptide comprises a variant or modified form of the binding domain.

4. The dendrimer according to claim 3 wherein the binding domain of the β integrin subunit has opposite end regions that bind to the MAP kinase and which are linked to each other by an intervening amino acid linker sequence that is not essential for binding with the MAP kinase.

5. The dendrimer according to claim 4 wherein one or more amino acids of the intervening amino acid sequence are deleted in the polypeptide compared to the binding domain of the β integrin subunit.

6. The dendrimer according to claim 5 wherein all of the amino acids in the intervening amino acid sequence are deleted in the polypeptide compared to the binding domain.

7. The dendrimer according to claim 5 wherein said opposite end regions of the binding domain remain unchanged in the polypeptide.

8. The dendrimer according to claim 3 wherein the polypeptide comprises or consists of the amino acid sequence represented by R/K×R/K*R/K−xx*x* NPL Y/F R/K, wherein each R/K is independently either arginine or lysine, Y/F is either tyrosine or phenylalanine, x is any amino acid, * is a hydrophobic amino acid, and − is an amino acid, wherein one or more amino acids of the sequence −xx*x* is optionally deleted.

9. The dendrimer according to claim 8 wherein the amino acid defined by − is a serine.

10. The dendrimer according to claim 8 wherein the amino acid defined by − is deleted or is an amino acid selected from the group consisting of threonine, tyrosine, asparagine and glutamine.

11. The dendrimer according to claim 8 wherein the polypeptide has an amino acid sequence defined by R/K×R/K*R/K NPL Y/F R/K.

12. The dendrimer according to claim 3 wherein the variant or modified form of the binding domain of the β integrin subunit includes at least 2 positively charged amino acid residues.

13. The dendrimer according to claim 1 wherein the polypeptide comprises, or consists of, an amino acid sequence selected from the group consisting of RSKAKWQTGTNPLYR (SEQ ID No: 2), RARAKWDTANNPLYK (SEQ ID No: 3), RSRARYEMASNPLYR (SEQ ID No: 4), KEKLKSQWNNDNPLFK (SEQ ID No: 5), RSKAKNPLYR (SEQ ID No: 6), RARAKNPLYK (SEQ ID No: 7), RSRARNPLYR (SEQ ID No: 8), and KEKLKNPLFK (SEQ ID No: 9).

14. The dendrimer according to claim 1 wherein the polypeptide is 10 amino acids in length or greater.

15. The dendrimer according to claim 1 wherein the dendrimer presents 10 units of the polypeptide.

16. The dendrimer according to claim 1 wherein the polypeptide comprises one or more D-amino acids and/or is N- or C-terminal protected against proteolytic degradation.

17. The dendrimer according to claim 1 wherein the β integrin subunit is selected from the group consisting of β2, β3, β5, and β6.

18. The dendrimer according to claim 1 wherein the MAP kinase is ERK2.

19. A pharmaceutical composition comprising a dendrimer as defined in claim 1 together with a pharmaceutically acceptable carrier.

20. A method for inhibiting growth of a cancer cell, comprising treating the cell with an effective amount of a dendrimer as defined in claim 1.

21. The method according to claim 20 being for prophylaxis or treatment of cancer in a mammal comprising administering the dendrimer to the mammal.

22. The method according to claim 20 wherein the β integrin subunit is expressed by cancer cells of the cancer.

23. The method according to claim 21 wherein the cancer is selected from breast, colon, gastric and prostate cancers.
